(19) United States
(12) Patent Application Publication
(10) Pub. No.: US 2023/0248430 A1
(43) Pub. Date: Aug. 10, 2023

(54) Optimally Integrated Generator Antenna System

(71) Applicant: Emblation Limited, Inglewood Alloa (GB)

(72) Inventors: Eamon McErlean, Inglewood Alloa (GB); Gary Beale, Inglewood Alloa (GB)

(73) Assignee: Emblation Limited, Inglewood Alloa (GB)

(21) Appl. No.: 18/012,208

(22) PCT Filed: Jun. 29, 2021
(86) PCT No.: PCT/GB2021/051638

§ 371 Date: Dec. 21, 2022

(30) Foreign Application Priority Data
Jul. 1, 2020 (GB) 2010057.4

(51) Int. Cl. A61B 18/18; H01Q 5/47; A61B 18/14; A61B 17/00

(52) U.S. Cl. CPC A61B 18/1815; H01Q 5/47; A61B 18/14; A61B 17/00; A61B 2018/1823; A61B 2018/1892; A61B 2017/00526; A61B 2018/00642

(57) ABSTRACT

A radio frequency (RF) or microwave energy applicator device (10) for applying radio frequency or microwave radiation to a target (22), the applicator (10) comprising: an energy generator module (12) for generating RF or microwave energy, wherein the energy generator module (12) comprises an energy output (16) for outputting said generated energy; a radiating structure (14) for radiating RF or microwave radiation to the target wherein the radiating structure (14) comprises an energy input (18), wherein the energy generator module (12) and the radiating structure (14) are coupled to provide the energy output (16) of the energy generator module (12) and the energy input (18) of the radiating structure (14) at a transmission interface (20); wherein the transmission interface (20) comprises at least one transmission feature comprising a size, dimension and/or shape selected so that at least part of the energy provided to the transmission interface (20) is transmitted to the radiating structure (18) and/or at least part of the energy provided to the transmission interface (20) is reflected.

### FIELD

The present invention relates to a radio frequency (RF) or microwave energy applicator device.

### BACKGROUND

In medical applications that utilise, for example, microwaves, the delivery of energy presents a number of technical challenges, the primary issue being the attenuation of energy from the point of creation to the point of delivery. In these applications in order to deliver the required amount of energy to facilitate a treatment, careful consideration must be made as to the delivery path and the associated losses.

In known energy ablation systems, the energy is generated by an energy generator and transmitted from the energy generator, via a connecting coaxial cable, to a radiating applicator that applies the energy to a treatment site of the tissue thereby transferring the energy into tissue. Known ablation systems have coaxial cabling between the energy generator and the applicator. FIG. 2 represents a simplified example of a known applicator: a RF or microwave power generator 2 is connected via a transmission line 3 to a radiating structure 4.

In known radiating applicators, a radiating element is, in use, positioned to be surrounded by the tissue, to penetrate or pierce the tissue or is placed in contact with the tissue. For these known systems, the typical standard treatment is to deliver energy for a treatment for a delivery period that lasts typically between 1 to 20 minutes to raise the temperature of the tissue greater than 43 to 45° C., for example, up to higher temperatures such as 60, 70 to 100° C. and beyond such that necrosis occurs within a desired ablation zone. In known energy ablation systems, the system may maintain or control the required level of delivered energy for the duration of the delivery period via amplitude or pulse width-modulated duty cycle control.

One undesired aspect of high frequency electromagnetic energy coaxial cabling is that energy may be lost within the cabling via heat along the length of the cable. Typically, the cabling may be designed to be both practical and short enough to ensure that sufficient energy is delivered to the treatment site. Interconnect cabling is typically 1 to 2 meters in length which may be acceptable for some applications as this length allows the generator to be located close to the patient and a 20-35% loss of energy is tolerated. The interconnecting cabling may form part of the treatment applicator or may be a lower loss reusable cable that connects to the higher loss (smaller) treatment applicator. Another disadvantage of high frequency coaxial cabling is that the cabling may be damaged through crushing or kinking which causes reflection or absorption of energy.

In response to one or more the above-described undesired aspects, two approaches have been proposed. The first is to place the energy generator system near to the treatment location. This may be achieved for example, by providing a microwave generator that is placed in a device or connector handle with a transmission line that links to the antenna radiating element to deposit the energy into the treatment location. Such an arrangement is described in US Patent Number: U.S. Pat. No. 9,039,693B2. However, in such a solution there may be a portion of transmission line that may lose energy in use before the energy arrives at the antenna.

A second approach is to place the energy generator in the same region as a radiation structure, as described in WO 2017/215972. In that work, a power amplifier/power source is located near to the radiating structure. A microwave generator is connected to a radiating structure via a separately identified transmission line. In that work the transmission line length, position and properties may be varied for tuning purposes. While tuning and impedance matching techniques may improve overall energy delivery efficiency these techniques can also contribute to power loss as the energy incurs attenuation via transmission line losses. Careful control of the electrical phase length of the adjoining transmission line or tuning stubs may also be required to maintain this. When the matching elements possess loss, a network designed to extract the most power from the generator may not necessarily deliver the most power to the load.

In both of the proposed approaches, some additional energy may have to be created by the energy generator to accommodate the overall path losses to ensure sufficient energy is delivered to the treatment site. In RF and microwave systems, any increased energy requirement may add complexity, bulk and expense to the system. Transmission lines may also add dimensional constraints with path losses adding to heating thereby absorbing useful energy.

In known applicators with transmission lines, standard radiating antenna may be designed to match to a feed reference impedance e.g. 50Ω. In most cases, antenna mismatch may be minimal for a broadband performance. In medical applications, the antenna may not always provide an optimal broadband match as tissue does not possess a universal dielectric constant as air does. In terms of a network cascade, a power generator may typically be designed to match to a 50Ω load impedance, the antenna may be designed to match to a 50Ω source impedance with both connected via a 50Ω transmission line. In theory, optimum power transfer for this arrangement may take place, however any mismatches that reflect and add or cancel, depending upon the phase properties of the transmission line may impact performance. It is known to vary the transmission line phase property (or electrical length) to, for example, improve the energy delivered or to cancel out unwanted reflection signals, however this method has limitations in that more than one performance attribute may be tuned simultaneously, resulting in a trade-off which may not be optimal. In addition, tuning by adding stubs or quarter-wave transformers may introduces further loss mechanisms.

Therefore, there is a need for a new RF or microwave energy applicator that may address at least one of the above disadvantages.

### SUMMARY

According to a first aspect, there is provided a radio frequency (RF) or microwave energy applicator device for applying radio frequency or microwave radiation to a target, the applicator comprising:
an energy generator module for generating RF or microwave energy, wherein the energy generator module comprises an energy output for outputting said generated energy;
a radiating structure for radiating RF or microwave radiation to the target wherein the radiating structure comprises an energy input,
wherein the energy generator module and the radiating structure are coupled to provide the energy output of the energy generator module and the energy input of the radiating structure at a transmission interface;
wherein the transmission interface comprises at least one transmission feature comprising a size, dimension and/or shape is selected so that at least part of the energy provided to the transmission interface is transmitted to the radiating structure and/or at least part of the energy provided to the transmission interface is reflected.

The energy output of the energy generator module and the energy input of the radiating structure may be coupled such that no variable structure is required for tuning between the energy output of the energy generator and the energy input of the radiating structure. The energy output of the energy generator module and the energy input of the radiating structure may be coupled such that no co-axial cable or phase variable structure or electrical length variable structure is provided between the energy output of the energy generator and the energy input of the radiating structure. The transmission interface may be such that there is substantially no extendable or variable transmission line between the energy generator and the radiating structure. The energy output and the energy input may be directly coupled.

The transmission feature may comprise a mismatch between the energy output of the energy generator module and the energy input of the radiating structure thereby to introduce a transmission inefficiency between the energy output of the energy generator and the energy input of the radiating structure.

The radiating structure may be a rigid structure and the energy generator module may be a rigid structure. The radiating structure and the energy generator module may be rigidly coupled together.

The device may comprise no flexible or extendable cabling, for example, no variable length co-axial cable, between the rigid energy generator module and the rigid radiating structure. The transmission interface may be between a first surface of the energy input and a first surface of the energy output. In addition, the transmission may also be provided between a second surface of the energy input and a second surface of the energy output. The transmission interface may lie, at least in part, in a plane substantially parallel to a propagation direction of the generated energy. The transmission interface may comprise a first part in a plane parallel to a propagation direction of the generated energy and a second part in a plane perpendicular to a propagation direction of the generated energy.

The radiating structure may comprise a radiating surface from which radiation is emitted and wherein the transmission interface provides the only interface between the energy generator module and the radiating surface.

The transmission feature may confer transmission and/or reflectance properties on the transmission interface. The transmission interface may permit a first desired portion of the energy provided to it to be transmitted. The transmission interface may reflect a second desired portion of the energy provided to it. The transmission interface may prevent transmission of a third desired portion of the energy provided to it.

By providing a microwave applicator in accordance with the first aspect, the microwave applicator may not require a cable or an extended transmission line between the energy generator module and the radiating structure. Therefore, a compact applicator may be provided. The energy generator module and the radiating structure may be coupled to provide an integrated applicator device.

At least one of the energy output of the energy generator module and the energy input of the radiating structure may be shaped and/or sized to form the transmission feature at the transmission interface.

The at least one transmission feature may comprise a discontinuity or mismatch between the energy output and the energy input. The at least one transmission feature may comprise a width and/or height of the energy input and/or a width and/or height of the energy output to provide a discontinuity between the width and/or height of the energy input and the width and/or height of the energy output.

The at least one transmission feature may comprise one or more of a slot, a gap, a protrusion in at least one of the energy output of the energy generator module and the energy input of the radiating structure.

The at least one of a size, dimension and/or shape may be selected to substantially maximise a measure of transmitted power from the energy generator module to the radiating structure and/or to substantially minimize a transmission loss through the transmission interface.

At least one design parameter for the radiating structure and/or the energy generator module may be selected together with the at least one of size, dimension and/or shape of the transmission feature to provide a desired degree of impedance match between the energy output and the energy input. At least one of the impedance of the energy output and/or the impedance of the energy input may not correspond to a standard impedance value, for example, an impedance value of 50Ω.

At least one design parameter for the radiating structure and/or the energy generator module may be selected to provide a desired degree of bandwidth match.

The at least one design parameter of the radiating structure and/or energy generator module may be selected to provide a substantially simultaneous impedance match between the radiating structure and a desired surface and between the radiating generator module and the energy generator module. The at least one design parameter may be selected such that, together with the transmission feature, a substantially system-wide conjugate match is achieved.

The at least one design parameter of the radiating structure may be in dependence on at least one of a property of a target to which the RF or microwave radiation is to be applied. The at least one design parameter of the radiating structure may be selected in dependence on at least one of: a volume of tissue to be treated, a property of tissue to be treated, a dielectric constant of tissue to be treated, a type of treatment.

The at least one design parameter may comprise a dimension, for example, a height, width, length or thickness of at least part of the energy generator module, for example, the energy output. The at least one design parameter may comprise a dimension, for example, a height, width, length or thickness of the radiating structure, for example the energy input. The at least one design parameter may comprise a length of the exposed distal portion of a conductor of the energy input or output. The at least one design parameter may comprise a length or phase property of the radiating structure. The at least one design parameter may comprise an offset distance between parts of the radiating structure. The at least one design parameter may comprise a gap between a radiating element of the radiating structure and an outer conductor.

The transmission feature may comprise an overlapping feature, for example, a step feature, such that at least part of the energy output and at least part of the energy input are at least closely coupled along an overlap length. The portion of energy transmitted and/or reflected may be in dependence on the overlap length.

At least part of a first surface of the energy output and at least part of a first surface of the energy input may be provided in direct contact along the overlap length. At least part of a second surface of the energy input may be provided in direct contact with at least part of a second surface of the energy input along the overlap length. The distance between the first surface and/or second surface of the energy output and the first surface and/or second surface of the energy input may be less than a pre-defined coupling distance. The pre-defined coupling may be less than 5 mm, or preferably less than 1 mm.

The overlap length may be a length in a direction parallel to the propagation direction of the generated energy. The overlap length may in a direction parallel to a longitudinal axis of the radiating structure and/or a longitudinal axis of the energy generator module.

The overlap length may be in the range 1 mm to 8 mm. The overlap length may be in the range 3 mm to 6 mm.

One of the energy output and the energy input may comprise a geometric feature, for example, a void, shaped and/or sized to engage and/or mate with a corresponding geometric feature of the other of the energy output and the energy input.

The transmission interface may comprise an interface between a microstrip structure and a co-axial structure. The energy input of the radiating structure and/or the energy output of the energy generator module may comprise a microstrip structure comprising a microstrip conductive element on a substrate. The energy input and/or output of the radiating structure may comprise a coaxial input structure comprising an inner conductor and an outer conductor.

The energy output of the energy generator module may comprise a first exposed length of a microstrip conductive element on a substrate and the energy input of the radiating structure comprises a second exposed length of an inner conductor of a coaxial structure such that when coupled, the first exposed length is provided at the second exposed length.

The at least one transmission feature may provide at least one conductive path between the energy generator module and the radiating structure.

The energy input of the radiating structure may comprise a rigid coaxial structure comprising an inner conductor and an outer conductor. The at least on design parameter may comprise a length and/or width of the rigid coaxial structure. The at least one design parameter may comprise a radius of the first conductor and/or a radius of the second conductor.

The energy output of the energy generator module may comprise a rigid microstrip structure comprising a microstrip conductive element provided on a substrate, and a ground layer. The at least one design parameter may comprise a thickness of the ground layer. The at least one design parameter may comprise a width and/or height of the substrate. The at least one design parameter may comprise a width and/or length and/or height of the microstrip conductive element.

At least part of the energy generator module and/or at least part of the radiating structure may be sized and/or shaped to fit the energy generator module together with the radiating structure such that, when fitted together, a conductive path is provided between the energy generator module and the radiating structure.

The transmission feature may further comprise an insulating portion at least partially surrounding the at least one conductive path, wherein the insulating portion is provided by at least part of the energy generator module and/or at least part of the radiating structure.

The device may further comprise a coupling mechanism for coupling the energy generator module and the radiating structure.

The coupling mechanism may comprise a mounting mechanism for mounting the radiating structure on a mounting portion of the energy generator module. The coupling mechanism may further comprise a securing mechanism for securing the radiating structure to the energy generator module. The coupling mechanism may comprise a screw or other fastening means. The screw of other fastening means may comprise a conductive material. The parts may be fixedly coupled so that the at least one of a size, dimension and/or shape is a fixed quantity.

The coupling mechanism may provide at least one electrical path between the radiating structure and a ground of the energy generator module via a portion of the coupling mechanism. The coupling mechanism may provide a first conductive path at an upper surface of the microwave generating module and a second conductive path at a lower surface of the microwave generating module.

The energy generator module may comprise a feedback mechanism configured to receive energy reflected by the radiating structure or a signal representative thereof. The one or more design parameters of the radiating structure may be selected such that the radiating structure reflects a desired portion of energy provided to so that feedback mechanism causes the energy generator module to generate more energy.

The radiating structure may comprise any suitable antenna, for example, a dipole antenna, a monopole antenna, a horn, a waveguide. The device may further comprise a housing. The energy generator module may comprise an amplifier stage and wherein the transmission interface comprises a secondary coupling between the power amplifier of the generator module and the radiating structure. The radiating structure may comprise a second order extracted pole unit (EPU) composed of a pair of mutual coupled resonant elements. The radiating structure may comprise one or more dissipative elements configured to dissipate excess heat into metallic or thermally conductive elements within the radiating structure. The device may further comprise a controller to control one or more operational parameters.

According to a second aspect there is provided a method of designing a RF or microwave energy applicator device comprising:
generating a model representative of at least a transmission interface between an energy generator module and a radiating structure, wherein the transmission interface comprises at least one transmission feature;
varying one or more parameters representative of the size, dimension and/or shape parameters of at least the at least one transmission feature to determine changes in the portion of energy reflected and/or transmitted at the transmission interface;
selecting values for the one or more design parameters corresponding to a desired portion of energy reflected and/or transmitted via the transmission interface.

The method may further comprise performing an optimisation process and/or iteratively selecting value for one or more design parameters and determining the effect on one or more operational parameters of the applicator device thereby to reach a target value of the one or more operational parameters.

The method may further comprise:
generating at least one further model representative of the interface between the radiating structure and a desired surface and combining the at least one further model with the model representative of at least the transmission interface, and selecting one or more design parameters of the radiating structure, the energy generator module and the transmission interface based on the combined model.

According to a third aspect there is provided a method of manufacturing a RF or microwave energy applicator device comprising:
providing an energy generator module comprising an energy output and a radiating structure comprising an energy input in accordance with one or more design parameters such that the energy generator module and the radiating structure comprises one or more transmission and/or reflection properties such that when the energy input and the energy output are coupled at a transmission interface, one or more transmission feature comprising a size, dimension and/or shape selected to transmit and/or reflect a desired portion of microwave energy provided to it from the energy generator module.

Features in one aspect may be applied as features in any other aspect, in any appropriate combination. For example, system features may be provided as method features or vice versa.

### BRIEF DESCRIPTION OF THE DRAWINGS

Embodiments will now be described by way of example only, and with reference to the accompanying drawings, of which:

FIG. 1 is a schematic diagram of a known RF or microwave energy applicator;

FIG. 2 is a schematic diagram of a microwave applicator, in accordance with embodiments;

FIG. 3(a) is a side view of the microwave applicator in accordance with an embodiment, the applicator comprising an energy generator module, radiating structure and transmission interface and FIG. 3(b) is an top view of the transmission interface and energy generator module;

FIG. 4 is a cross-sectional view of the transmission interface of the microwave applicator;

FIG. 5 is a perspective view of the transmission interface of the microwave applicator, and

FIG. 6 is a photographic representation of the microwave applicator in accordance with an embodiment.

FIGS. 7(a) and 7(b) are plots illustrating the variation of scattering parameters in dependence on frequency and a design parameter;

FIGS. 8(a), 8(b) and 8(c) are screenshots of a graphical interface used in the design of the integrated applicator;

FIG. 9 is a diagrammatic illustration of the standard theory of a 2-port matching network;

FIG. 10 is a schematic representation of a theoretical framework underpinning the design of the microwave applicator;

FIG. 11 is a schematic illustration of a cascaded S-parameter model, and

FIG. 12 is an illustration of a coupling matrix representation.

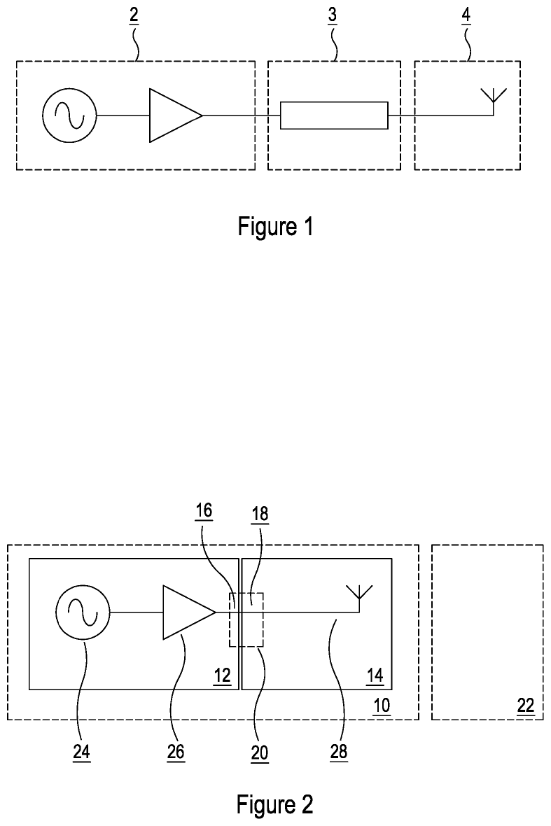

### DETAILED DESCRIPTION

A radio frequency (RF) or microwave energy applicator and a method of designing such an applicator is described. The apparatus and methods described herein are applicable for both industrial and medical applications. In the following, an electromagnetic energy generator module is described that is configured to generate energy in the frequency range of 1 KHz to 300 GHz.

FIG. 2 is a schematic diagram of the integrated applicator 10, in accordance with embodiments, which is referred to, for brevity, as an applicator 10. The applicator 10 has a microwave energy generator module 12, herein referred to, for brevity, as simply the energy generator module and a radiating structure 14. The energy generator module 12 has an energy output 16 for outputting generated energy. The radiating structure 14 has an energy input 18 for receiving energy.

It will be understood that, while the present embodiment is described with respect to generation and delivery of microwave energy, in other embodiments in which RF frequency radiation is used the same principles are used.

The energy generator module 12 has microwave generating circuitry. In the present embodiment, the energy generator module 12 is a microwave energy generator module and has a signal generator or oscillator (VCO) 24 and an amplifier stage 26. In some embodiments, the components are such that the microwaves generated are suitable for application to a particular surface or, more generally, a particular target 22, for example, tissue to be treated. The radiating structure 14 is configured to emit electromagnetic radiation that will be received optimally by the target 22. The radiating structure 14 emits radiation, for example from a radiating surface. In the present embodiment, the radiating structure is comprises antenna 28.

Between the energy output 16 of the energy generator module 12 and the energy, input 18 of the radiating structure 14 there is a transmission interface 20. Energy that is output from the energy output 16 of the energy generator module 12 is provided to the energy input 18 via the transmission interface 20. The transmission interface 20 is formed such that it has a transmission feature having at least one of a size; dimension and/or shape selected to control or otherwise modify the transmission and/or reflection properties of the transmission interface 20. Controlling or modification of the transmission and/or reflection properties of the transmission interface 20 may contribute to an optimization of the performance of the applicator 10. For example, the power transmitted through the interface 20 may be maximised or transmission power losses via the interface 20 may be minimized.

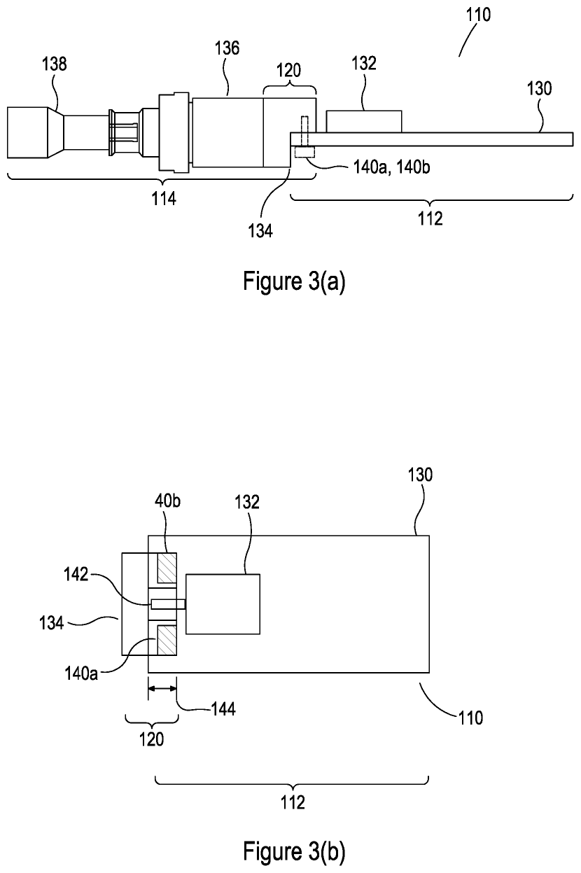

It will be understood that a number of different transmission features may provide desired transmission/reflectance properties for the transmission interface 20. A suitable transmission feature has a shape, size or dimension that may be varied during a design process to allow the effect of the variation to be assessed and therefore allowing the design to be optimized for a specific requirement. This may allow for an optimal operation of the applicator 10, in use. An embodiment of the applicator with a particular transmission feature is described with reference to FIGS. 3 to 6.

In use, microwave energy is generated by the energy generator module 12 and provided to the transmission interface 20. In accordance with the transmission/reflectance properties conferred on the transmission interface 20 by the transmission feature, the transmission interface 20 receives the energy provided to it and, permits a first desired portion of energy provided to it from the energy generator module 12 to be transmitted to the radiating structure and/or reflects a second desired portion of energy provided to it back to the energy generator module 12. The transmitted energy is provided to the radiating structure 14 to be radiated by antenna 28.

In some embodiments, the energy generator module 12 and the radiating structure 14 may be known off-the-shelf components, for example, components that are tuned to have a standard impedance or other standard properties. However, it will be understood that, in some embodiments, at least one of these parts may be designed to be a bespoke component and manufactured to have particular desired properties.

In the present embodiment, the radiating structure 14 is designed such that, when integrated with the energy generator module 12, the radiating structure 14 presents ideal output impedance characteristics to the energy generator module 12. Likewise, the radiating structure 14 is designed to possess the optimal required input impedance characteristics. As described in the following, the process of integrating the two parts may comprise selecting one or more values for design parameters of the radiating structure 14 and/or the energy generator module 10 to optimize one of more properties of the energy transferred therebetween or a related parameter. In some embodiments, in addition to selecting one or more design parameters of the radiating structure 14 and energy generator module 10, the arrangement may further also incorporate properties that are observed when the radiating structure 14 is presented with its ideal or typical target media. When the designed parts of the integrated applicator are fully integrated into a signal unit, they may be considered to be arranged in a balanced configuration and therefore the requirement for additional separate tuning elements, matching networks, fractions of wavelength or phase length transmission line tuning elements is reduced or eliminated. This may provide size and performance advantages.

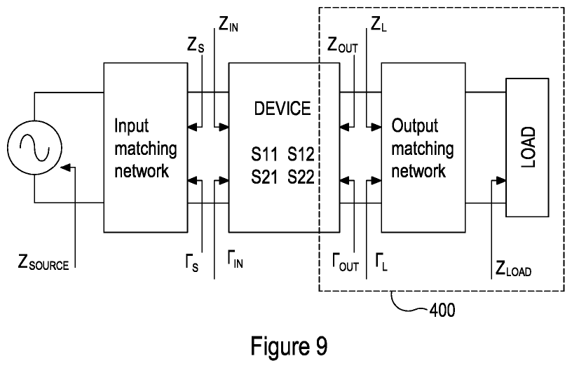

The design of the parts of the applicator 10 may be made in accordance with a theoretical framework. While different theoretical frameworks/models may be used in the design of the integrated applicator, a known theoretical framework includes a framework based on using scattering or S parameter models in which different interfaces between different parts of the applicator are modelled and combined using S parameter models. Further details regarding the theoretical framework is provided with reference to FIGS. 9 to 12.

FIGS. 3 to 6 depicts an embodiment of the integrated applicator. FIGS. 3(a) and 3(b) depict an integrated applicator, also referred to simply as the applicator 110, in accordance with the present embodiment. FIG. 3(a) shows a side view of the applicator 110. FIG. 3(a) shows the applicator 110 having an energy generator module 112 also referred to as an energy generator module, which in the present embodiment is a microwave energy generator module, and a radiating structure 114. The applicator 110 also has a transmission interface 120. FIG. 3(b) shows a top view of the energy generator module 112 and transmission interface 120. FIG. 3(b) depicts part of the radiating structure 114.

In further detail, in the present embodiment, the energy generator module 112 has a printed circuit board (PCB) 130, upon which is mounted microwave power generating devices or circuitry 132. The energy generator module 112 is a rigid structure and the radiating structure 114 is a rigid structure. The energy generator module 112 is rigidly coupled to the radiating structure 114 such that the output of the energy generator module 112 is provided at a transmission interface 120 and such that the input of the radiating structure 114 is provided at the transmission interface 120. In the present embodiment, the transmission interface 120 and its transmission features are formed by parts of the energy generator module 112 and the radiating structure 114. This coupling may also facilitate the transfer of thermal energy from the PCB conductive substrate/thermal heatsink 162 into the radiating structure to provide additional heatsinking.

The radiating structure 114 has a coaxial input portion 134, which is a rigid structure and may be referred to as a coaxial input structure. The radiating structure 114 also has a coaxial to waveguide feed section 136 and a waveguide 138. The coaxial to waveguide feed section 136 has a receptacle for receiving and holding the waveguide 138. The waveguide 138 is placed into the receptacle, which maintains electrical continuity to the waveguide ground plane using a cylindrical arrangement of sprung metallic fingers, flared to accept the waveguide 138.

As depicted in FIGS. 3(a) and 3(b), in the present embodiment, the radiating structure 114 is mounted to the PCB 130 and securely held in a mounted position by two bolts 140a and 140b. FIG. 3(a) shows first bolt 140a and FIG. 3(b) shows both first bolt 140a and second bolt 140b. Corresponding pairs of recesses are provided in the PCB 130 and coaxial input portion 134 of the radiating structure 114. For each of the pair of bolts, a first recess is provided in PCB 130 and a second recess is provided in the PCB 130. The recesses are provided, such that for each bolt, a pair of aligned recesses are presented for the bolt to pass through thereby mechanically securing the energy generator module 112 and radiating structure 114 together. It will be understood that other securing mechanisms may be used in other embodiments to secure the radiating structure 114 and energy generator module 112 together.

FIG. 3(b) shows a top view of the applicator 110, with the coaxial input portion 134 of the radiating structure 114 shown. Also shown in FIG. 3(b) is a microstrip element 142 of the energy generator module 112 which provides an output for generated microwave energy from the module. The microstrip element 142 is printed on the upper surface of the printed circuit board 130. FIG. 3(b) also shows bolts 140a and 140b.

In the present embodiment, the energy output of the energy generator 112 comprises a microstrip structure of which the microstrip element 142 forms a part. In the present embodiment, the energy input of the radiating structure 114 comprises the coaxial input portion 134 and its respective elements. The transmission interface 120, its transmission features and the energy input and outputs provided at the transmission interface 120 are described in further detail in the following, for the present embodiment.

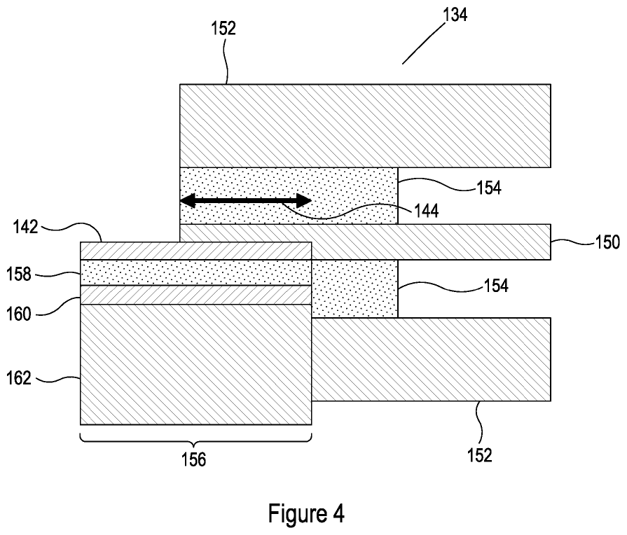

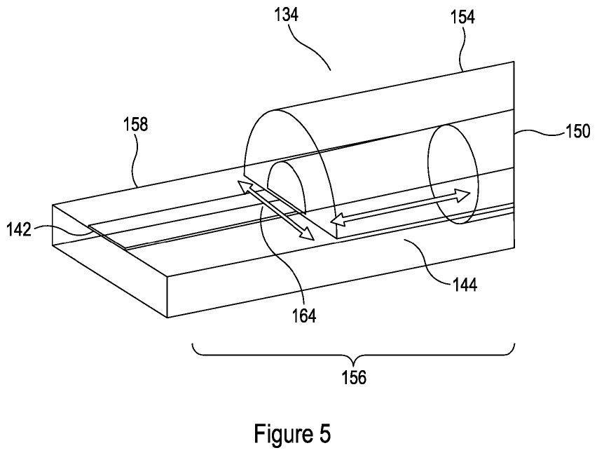

As can be seen from FIGS. 3(a) and 3(b) and as descried in further detail with reference to FIGS. 4 and 5, the radiating structure 114, in particular the coaxial input portion 134, and the energy generator module 112, in particular, the microstrip element 142 together form a transmission feature at the transmission interface 120. In the present embodiment, the transmission feature 120 is a step feature characterised by an overlap length 144. It will be understood that, while in the present embodiment, the transmission feature 120 is a step feature, alternative transmission features may be implemented at the transmission interface 120. Alternative transmission features may include tapered or gradual transition features. In further detail, in the present embodiment, the step feature provides an overlap between the radiating structure 114, in particular the coaxial input portion 134 that provides an energy input to the radiating structure 114 and the energy generator module 112, in particular the microstrip element 142, which forms part of an energy output for the energy generator module 112.

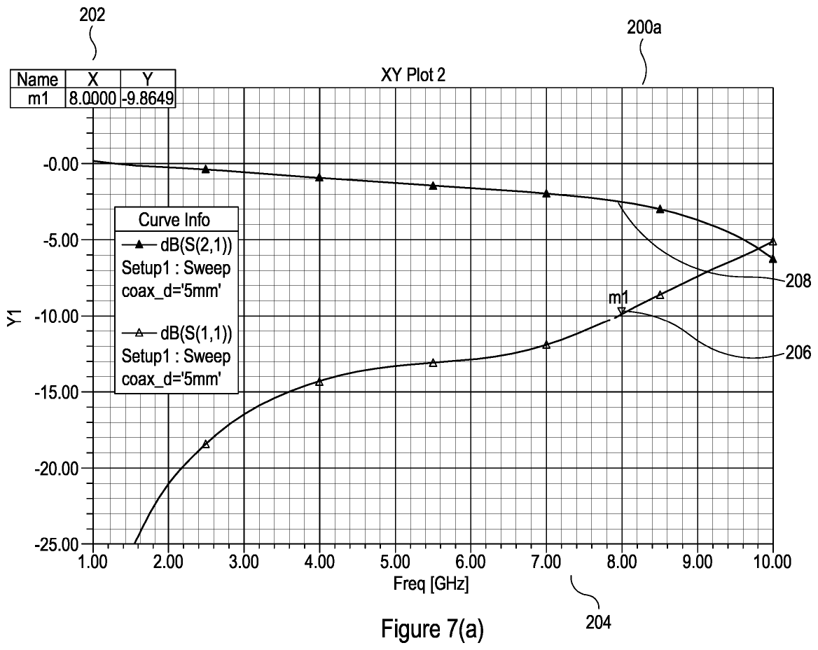

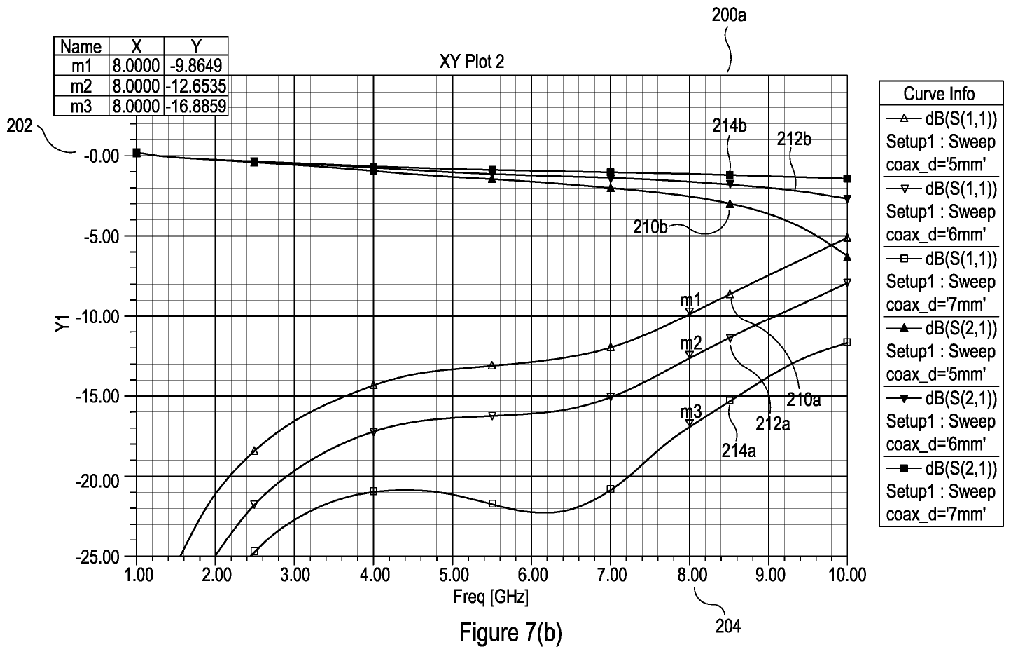

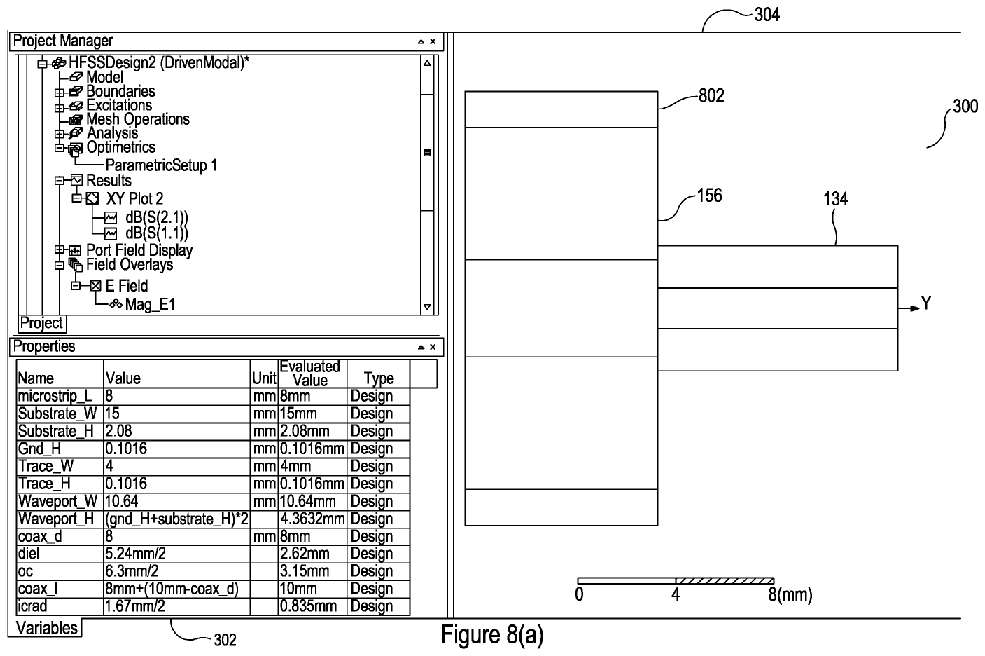

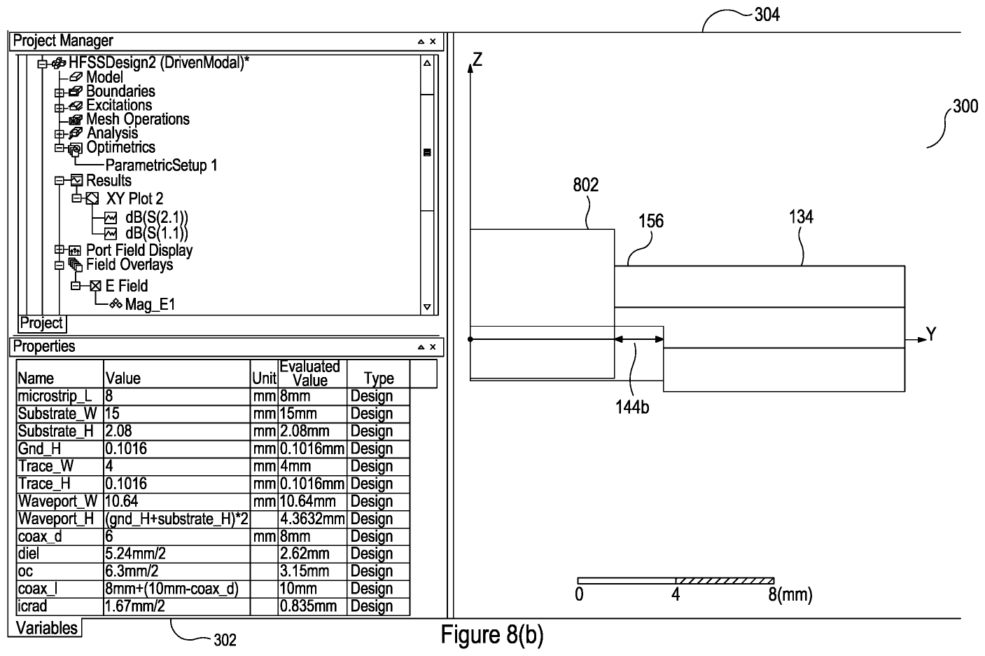

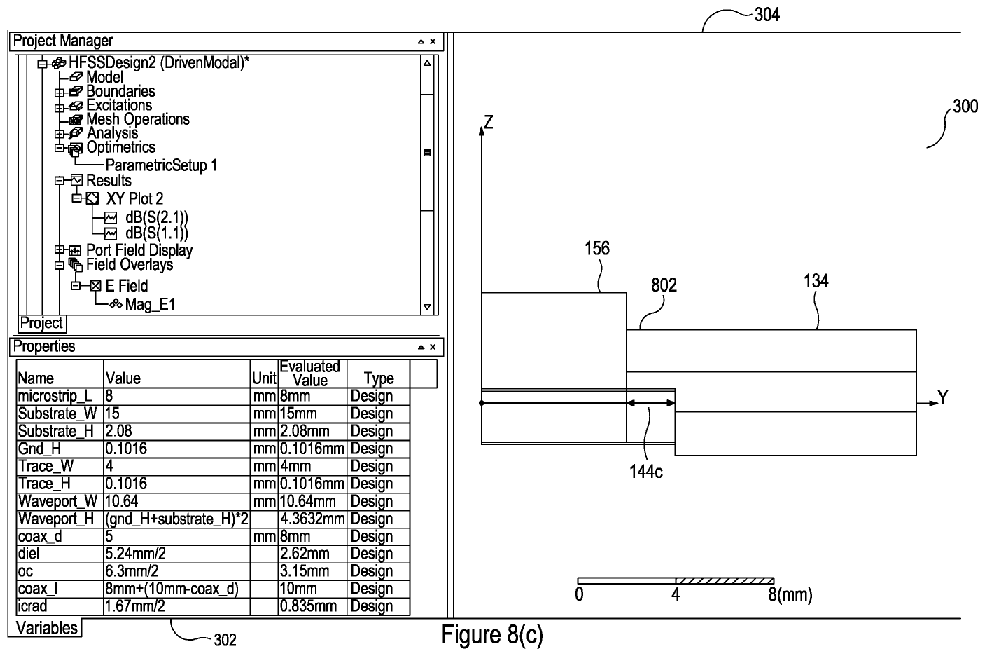

As described in detail with reference to FIGS. 7 and 8, the overlap length 144 characterising the step feature may have different values in different embodiments. Variation of the overlap length 144 may control the reflection and transmission characteristics of the transmission interface 120. In the present embodiment, the overlap length is 3 mm, however, this could be in a range between 1 mm to 8 mm, or for example 3 mm to 6mm. The overlap length is in a direction substantial parallel to the propagation of the energy generated by the energy generator module 112. The overlap length is parallel to a longitudinal axis of the radiating structure 114 and the energy generator module 112.

Due to the presence of the transmission interface 120, no flexible extendable transmission line, for example, no variable length co-axial cabling is required between the energy generator module 112 and the radiating structure 114.

In the present embodiment ground plane continuity is provided by including top ground plane connections to the radiating surface or antenna of the radiating structure 114. The conductive bolts 140a, 140b may also mate with an exposed ground plane on the underside of the PCB 130 for an additional ground plane connection.

FIG. 4 is a cross-sectional illustration of the transmission interface 120. FIG. 4 shows parts of the coaxial input portion 134. The coxial input portion 134 has an inner conductor 150 coaxial with an outer conductor 152. At the input end of the coaxial input portion 134 (the end provided proximal to the coupled energy generator module 112) a coaxial dielectric material 154 substantially fills the space between the inner conductor 150 and the outer conductor 152. The coaxial dielectric material 154 may also be referred to as an insulating material. The coaxial dielectric material 154 holds the inner conductor 150 and outer conductor 152 in place and electrically isolates the inner conductor 150 from the the outer conductor 152.

FIG. 4 shows the microstrip structure 156 of the energy generator module 112 in further detail. The microstrip structure 156 has a layered structure comprising of an upper conductor layer corresponding to the microstrip element 142, provided on a dielectric layer 158 and a ground plane layer 160. These layers are provided on a conductive substrate/thermal heatsink 162.

FIG. 4 shows a cross-sectional view of the step feature between the microstrip structure 156 and the coaxial input portion 134. In FIG. 4, cross-shaded elements are conductive metals and dot shaded elements are dielectric insulators. As can be seen from FIG. 4, the step feature is created by removing a portion of the coaxial input portion 134, in particular, the inner conductor 150, the outer conductor 152, the coaxial dielectric material 154 to create a void in the coaxial input portion 134, the void having an upper surface in the inner conductor 150 and a side surface of inner conductor 150, coaxial dielectric material 154 and outer conductor 152. The coaxial input portion 134 and microstrip structure 156 are then arranged such that the distal portion of the microstrip structure 156 is placed into the void. When in place, at least part of the upper surface of the microstrip structure 156 abuts an upper surface of the void and the side surface of the microstrip structure 156 abuts the side surface of the void such that the microstrip structure 156 is fitted into the void of the coaxial input portion 134. When arranged in postion, at least part of the microstrip element 142 of the microstrip structure 156 is in direct contact with the inner conductor 150 along a length corresponding to the overlap length 144.

In the present embodiment, as can be seen from FIG. 4, as described above, a first surface of the coaxial input portion 134 is in direct contact with a first surface of the microstrip structure 156 and a second surface of the coaxial input portion 134 is in direct contact with a second surface of the microstrip structure 156, wherein the second surfaces are perpendicular to the first surfaces. It will be understood that, in some embodiments, the first and/or second surfaces are not provided in direct contact but rather provided at a separation equal to a pre-defined coupling distance, for example, 1 mm.

FIG. 5 depicts a further, perspective view of the transmission interface 120, in particular FIG. 5 shows the step feature described with reference to FIGS. 3 and 4. As can be seen from FIG. 5, the step feature comprises both an overlap width 164 corresponding to the radius of the proximal end of the coaxial input portion 134 and the overlap length 144. The step feature also has a height. The dielectric layer 158 of the microstrip structure 156 is exposed at the upper surface so that the upper surface of the printed circuit board has both a conductive portion and an insulating portion. The coaxial input portion 134 has a width such that the dielectric material 154 of the coaxial input portion 134 is in contact with part of the exposed dielectric layer 158 of the microstrip structure 156. The step feature thus comprises a dielectric or insulting surrounding for the conductive microstrip element 142 formed by dielectric material 154 of the coaxial input portion 134 and dielectric layer 158. The surround substantially surrounds the conductive microstrip element 142 of the microstrip structure 156 thereby acting as an insulator. It is also noted, from FIG. 5, that there is no insulating dielectric layer between the contacts i.e. a direct conductive connection is made.

The above step feature is just one example of a transmission feature that can be provided at the transmission interface 120. The step feature is an example of a coaxial step discontinuity used to interface with a microstrip trace on a PCB. The microstrip trace is intended to be as short as possible and functions as a connection to the antenna and is not intended to be tunable transmission line.

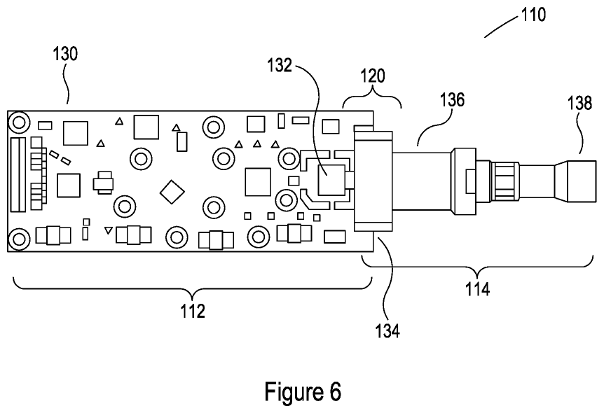

FIG. 6 is a photographic representation of the integrated applicator 110. FIG. 6 depicts a number of elements of the integrated applicator 110 described with reference to FIGS. 3, 4 and 5.

In the present embodiment, the radiating structure 114 is mounted directly onto the energy generator module 112 and is secured from beneath using bolts 140a, 140b as depicted in FIGS. 3(a) and 3(b). It will be understood that the electrical connections of the device can include, for example, direct contact, solder, conductive epoxy or PariPoser® anisotropic elastomer material. Once secured in position it will be understood that in the present embodiment, the radiating structure 114 and energy generator module 112 are fixed relative to each other, and are not moveable i.e. do not slide. The integrated device 110 offers the advantage that there is no requirement for moveable or tuneable elements, as all optimisation is achieved during the design stage, in which values for one or more design parameters are selected.

In the above-described embodiments, a step feature is described as a non-limiting example of a transmission feature at the transmission interface. However, it will be understood that the transmission feature(s) may comprise any form of discontinuity at the transmission interface between the energy output and the energy input. As a further non-limiting example, a width of the energy input of the radiating structure and/or the energy output of the energy generator module may be selected such that there is a mismatch in widths thereby providing an interruption or discontinuity between the energy output and energy input. Similar mismatches in other dimensions may be designed, for example, the height of the energy input and output. Mismatches in shapes can also be implemented, for example, a tapered structure may be selected. The transmission feature may comprise a mismatch between the energy output and energy input, for example, in size, shape or other dimensions, or other discontinuity, thereby to introduce a transmission inefficiency at the transmission interface.

As a further example, the at least one transmission feature may alternatively or additionally include other features that provide discontinuities at the transmission interface, for example, a slot or a gap or a protrusion in at least one of the energy output of the energy.

With reference to the above-described embodiment in which a coaxial structure is coupled to a microstrip structure, a discontinuity may be provided in the microstrip or the coaxial structure, or both. For the microstrip, any region that was too thin or too wide could cause a discontinuity. In terms of the coaxial structure, in the above-described embodiment a discontinuity was introduced in the inner conductor. However, it will be understood that the transmission feature may comprise at least one of the following non-limiting examples: a change in a coaxial ratio (the ratio between the inner conductor and outer conductor radius), a longitudinal slot in the coaxial outer conductor, a radial slot gap in the coaxial outer conductor or a perturbation or protrusion in the outer conductor. A conductive pin or washer could provide a protrusion in the outer conductor.

As described in the following, components of the intergrated applicator are optimized during a design process. The overlap feature is one of a number of antenna design factors that may be be used to adjust performance during design.

FIGS. 7(a) and 7(b) are plots (200a, 200b) depicting simulated values for S matrix parameters. In particular, FIGS. 7(a) and 7(b) illustrate the variation of the S matrix parameters S₁₁ and S₂₁ as a function of radiation frequency. S₁₁ represents the amount of power reflected from the cascaded radiating structure and may be referred to as the reflection coefficient. If this parameter is zero, then all power is reflected from the radiating structure and nothing is radiating. S₂₁ represents the transmission loss and conversely the lower this loss the higher the energy transferred. The Y-axis 202 for both plots of FIGS. 7(a) and 7(b) is a log-scale and have units of dB. The X-axis 204 for both plots is frequency.

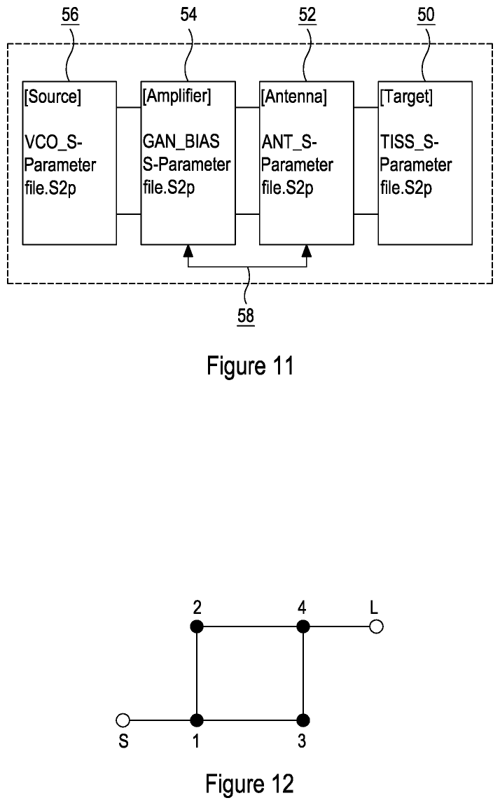

In the present embodiment, the parameters S₁₁ and S₁₂ take into account target/tissue properties. In particular, parameter S₁₁ n relation to FIGS. 7(a) and 7(b) relate to a cascade of antenna and target/tissue properties (i.e. references 50 and 52 of FIG. 11). In other embodiments in which a full cascaded model is implemented, the parameters also take into account the properties of the amplifier and source models (references 54 and 56 of FIG. 11).

A first plotted line 206 in FIG. 7(a) is representative of S₁₁ as function of frequency. A second plotted line 208 in FIG. 7(b) is representative of S₂₁ as a function of frequency.

For parameter S₁₁, it will be understood that, in some embodiments, anything that has values below −10 dB may be considered as acceptable. For parameter S₂₁, it will be understood that, in some embodiments, a transmission loss close to zero may be desirable. In other embodiments, a proportion of reflected energy may be desirable.

During the design process, values for design parameters of the coupling interface are selected and varied to simulate the effect of variation of the parameter values on the S-matrix parameters. In FIG. 7(b), values for S-parameters are plotted to illustrate the effect of variation of the overlap length (illustrated as numeral 144 in, for example, FIG. 4). The overlap length relates to the size of overlap between the co-axial input portion 134 and the microstrip structure 156. It will be understood that the overlap length may or may not be varied directly; it may also be varied through one or more other parameters on which the overlap length is dependent. In this embodiment, the overlap length is dependent on the coaxial distance parameter (coax_d). For each value of the parameter being varied, in this case, the coaxial distance (coax_d) there is a pair of plotted lines corresponding to the values for S₁₁ and S₂₁. FIG. 7(b) shows pairs of plotted lines for values for the coaxial distance for 5 mm, 6 mm and 7 mm (corresponding to overlap lengths of 3 mm, 2 mm and 1 mm, respectively).

For the S₁₁ parameter, plotted lines 210a, 212a and 214a correspond selection of the value for the coaxial distance parameter to be 5 mm, 6 mm and 7 mm, respectively. For the S₂₁ parameter, plotted lines 210b, 212b and 214b correspond to selection of the value for the coaxial parameter to be 5 mm, 6 mm and 7 mm, respectively.

FIGS. 8(a), 8(b) and 8(c) are screenshots of a graphical interface 300 used for designing the applicator. On the left side of FIGS. 8(a), 8(b) and 8(c) is a user interface panel 302 that allows a user to select different values for design parameters. These parameters include:
microstrip_L (microstrip conductive element, or tab, length)
Substrate_W (substrate width)
Substrate_H (substrate height)
Gnd_H (ground layer thickness)
Trace_W (microstrip conductive element width)
Trace_H (microstrip conductive element thickness)
Waveport_W (port width)
Waveport_H (port height)
Coax_d (coax/microstrip relative position)
Diel (coaxial dielectric radius)
Oc (outer conductor radius)
Coax_I (coax length beyond the overlap)
Icrad. (inner conductor radius)

The wave port height and width are only relevant to the modelling software and are not physical features. These were arbitrarily chosen (approximately 2 times the substrate height and approximately ⅔ of the substrate width).

On the right hand side of FIGS. 8(a), 8(b) and 8(c) is a viewing panel 304 for viewing a graphical representation of the applicator. FIG. 8(a) shows an overhead (top down) graphical representation of a view of a first simulated transmission interface between the coaxial input structure 134 and the microstrip structure 156.

FIGS. 8(b) and 8(c) show side views of a second and third simulated transmission interface, respectively, between the coaxial input portion 134 and the microstrip structure 156. Different values for the overlap length have been selected. In FIG. 8(a), the first simulated transmission interface has an overlap length of 0 mm (corresponding to a coaxial distance parameter of 8 mm). In FIG. 8(b), a second simulated transmission interface is depicted having an overlap length 144b of 2 mm (coaxial distance of 6 mm). In FIG. 8(c) a third simulated transmission interface is depicted that has an overlap length 144c of 3 mm (corresponding to a coaxial distance of 5 mm).

In these embodiments, only a single design parameter is varied, however, it will be understood that in other embodiments, more than one design parameter may be varied and/or selected.

The coaxial distance is related to the overlap length (the size of the step feature). In particular, in the present embodiment, the microstrip element 142 is retained at a fixed length (8 mm) and the parameter of coaxial distance (the distance between a first end of this fixed length and the distal end of the microstrip structure 156). It will be understood that selection of this parameter determines the size of the overlap length. In particular, in FIG. 8(a), the value of this parameter is 8 mm which is equal to the microstrip length parameter (microstrip_L) and therefore there is no overlap (overlap length is 0 mm). In FIG. 8(b), the value of this parameter is set to 6 mm which is 2 mm less than the microstrip length parameter of 8 mm (microstrip_L) and therefore there is an overlap (overlap length is 2 mm). In FIG. 8(c), the value of this parameter is set to 5 mm which is 3 mm less than the microstrip length parameter of 8 mm (microstrip_L) and therefore there is an overlap (overlap length is 3 mm).

In FIGS. 8(a), 8(b) and 8(c) a radiating boundary is modelled as a boundary box 802. The software assumes all other regions are perfect conductors (metal) so the model describes a coax inside a block of metal that has the variable step introduced to fit onto a PCB. Boundary box 802 is an approximation of radiation into free-space and is not a physical 3D feature of the model but rather provided as part of the modelling process.

In addition to the design of the transmission interface, further design parameters of the radiating structure and/or energy generator module or components thereof may be selected to control performance of the applicator. In known applicators, an antenna may be designed to impedance match to a 50 ohm transmission line and the energy generator module may be designed to impedance match to a 50 ohm transmission line. In such applicators, matching networks and other tuning elements are provided to compensate for mismatches between the components. In the present embodiments, the components are designed with reference to an underlying model i.e. taking into account the operation of the other components and the application target, such that when the components are plugged together the devices operate optimally.

For such a method, it has been found that there may be advantages in an integrated applicator that uses a radiating structure or a part thereof, for example, an antenna that is designed to have an input or other part that causes the antenna to reject energy. Such an antenna may be considered to provide what may be classed as sub-optimal performance when considered in other systems. In the integrated applicator, the amplifier of the generator module receives feedback from the antenna representative of the rejected energy and, in response to this feedback, causes further energy to be transmitted to the antenna.

It will be understood that the step discontinuity in the present embodiment does not alter the overall electrical length (path phase) and operates at 8 GHz within dimensions less than ¼ of a wavelength for a guided wave in the microstrip. For the following model parameters: dielectric constant of the printed circuit board (Er) of 4.4, a microstrip trace width (W) of 4 mm and a board height (H) of 2 mm (thickness), the calculated ¼ wavelength in the board is 5.125 mm. It will be understood that the dielectric compresses the electromagnetic wavelength compared to the equivalent free space wavelength. These dimensions are within one tenth of a wavelength and cannot be considered to constitute tuning as the discontinuity within this region creates a deliberate mismatch and adjustable level of loss that can be utilised.

The design of the parts of the applicator may be made in accordance with a theoretical framework. Further comments on the theoretical framework are provided in the following.

As discussed above, the design is such that additional matching networks may be avoided. Matching networks are often used for modelling applicators. An example of a two-port matching network arrangement is illustrated in FIG. 9. In this standard theory, a device with full 2-port S-parameters can be matched both to a generator (Z_{SOURCE}) and to a load (Z_{LOAD}) by means of Input (Γ_{S}/Γ_{IN}) and output (Γ_{L}/F_{OUT}) matching networks. In this theory, the output section 400 could represent an antenna with a tissue dielectric acting as the load. In this description, Z is impedance and Γ is a reflection coefficient.

Underpinning the present embodiments, is a concept similar to the concept of a conjugate match, the condition for maximum power delivery to a load, in which the impedance seen looking to the load at a point in a transmission line is the complex conjugate of that seen looking to the source. A conjugate match states that a maximum power is transferred between a source (like a transmitter) and a load (like an antenna), when the source impedance is the complex conjugate of the load impedance. The design principle followed is different to a single-end conjugate match and in principle follows Everitt's conjugate match theorem for lossless networks which states that if a conjugate match exists at any port in the cascade, then a conjugate match exists at every port in the cascade, including the input and output ports connected to the source and load with all available power is delivered to the load.

However, in reality transmission networks are not lossless, and although in theory a system-wide conjugate match in a network comprising lossy elements might be mathematically possible in practical terms the best solution is maximum power transfer which traditionally requires consideration of matching in both directions to ensure optimal power transfer. By minimising losses in the matching networks and by considering the quality factor, Q of load and source elements the closest approximation to a near system-wide conjugate match may be achieved.

As described above, in accordance with embodiments, no separate external matching networks or tuneable transmission lines are required for the integrated applicator. The radiating structure (or antenna element) is designed to bilaterally satisfy the matching requirement in addition to the radiating requirement by providing a very low-loss matching network function in each direction between the final target and the energy generator module or power source. In the present embodiment, the transmission line path from the energy source to the treatment applicator may be eliminated thereby reducing losses that would occur in the system via this transmission line and the additional energy required. This may lead to an improvement in efficiency.

In instances where the energy generator module or power source has an un-matched RF/Microwave transistor which has its own particular scattering parameters the same optimisation can be achieved by judiciously utilising a specific antenna-to-target mismatch in combination with antenna phase properties to present the desired complex reactive impedance as required the RF/Microwave transistor. In this way both elements can be co-designed as a single integrated energy transmission network.

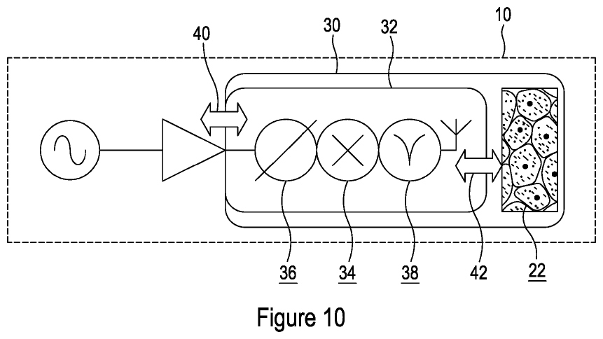

FIG. 10 shows a schematic diagram illustrating the theoretical framework underpinning a design process of the integrated applicator, in accordance with embodiments. FIG. 10 illustrates the different interfaces that may be considered when designing the applicator 10. In this framework, scattering matrices for different interfaces of the applicator are described.

FIG. 10 shows a representation of the integrated applicator 10. FIG. 10 depicts a combined antenna and tissue S-parameter model 30. The antenna and tissue S-parameter model 30 has an antenna element 32 (provided as part of the radiating structure) and certain properties of the antenna element 32 are represented in FIG. 10, including match 34, phase 36 and resonant bandwidth (Quality-factor) 38.

In FIG. 10, a generator-antenna interface model 40 is represented. In FIG. 10, the output matching networks (Γ_{L}/Γ_{OUT}) from theory are represented by the designed interaction 42 between the antenna and tissue 22 in combination with the interaction between generator and antenna 40.

Calculation and/or determination of design parameters may be implemented using a cascaded design approach. In this illustration, each S-parameter model is cascaded or otherwise combined to form an overall model of the integrated applicator.

In this example, the dielectric properties of the tissue target (which are either measured/sampled or simulated) are represented by 50. The tissue model is cascaded with a baseline S-parameter model for the antenna 52. In some embodiments, the combined network of tissue model 50 and antenna model 52 can then be optimised to present the desired impedance to the preceding stages: amplifier stage 54 and generator source stage 56 thereby to deliver the optimum energy to the tissue by adapting combined antenna/tissue attributes of match 34, phase 36 and resonant bandwidth (Quality-factor) 38 in the antenna model.

The S-parameter models may represent simple numerical cascaded S-matrix models or may also be hierarchically formed using or including hybrid combinations of S-parameter models and simulation S-parameter outputs of full-wave 3D solvers e.g. HFSS, XFdtd, COMSOL Multiphysics, FEKO etc. These 3D solvers can include complex electromagnetic interactions between each stage therefore one or more stages may be included in a 3D model that may be cascaded with an S-matrix model in the same or in another circuit-level simulator e.g. Microwave Office, Sonnet, ADS etc. S-matrix models, Y-matrix models or Z-matrix models or any combination therefore may be used depending upon the simulator used.

In addition, cross coupling of energy 58 from the antenna stage 52 to the amplifier stage 54, may also be employed to optimise the design further. This energy may be coupled directly e.g. cavity mode cross-coupling or indirectly by parasitic coupling. This method provides further options to employ finite transmission zeros which can be utilised to improve bandwidth or feedback to increase amplifier efficiency. This technique can also be achieved by loading the input of the antenna with a second-order extracted-pole unit (EPU) composed of a pair of mutual coupled resonators negating the need for physical cross-coupling. This can be realised by utilising stepped cross-sections or tab-cross feeds in the case of waveguide fed antennas.

In this regard, the overall design can also be treated in terms of coupling matrices. FIG. 12 represents coupling matrices, may employ coupling matrix synthesis methods to achieve the desired overall performance.

By implementing this invention, the design can be made more efficient, more compact and can eliminate the requirement for tuneable transmission lines, tuning stubs or other similarly physically distributed (or electronically or mechanically actuated) tuning arrangements that would have been necessary to improve efficiency.

In terms of fabrication, the integrated generator/antenna may be constructed from lightweight materials to permit a reduction in the mass. In some embodiments, the applicator may also take advantage of the integrated construction to dissipate excess heat into metallic or thermally conductive elements within the antenna to reduce size further. The integrated generator/antenna may also have particular thermal interface points that could mate with heatsinking elements e.g. Cu—Cu brackets or pyrolytic carbon or thermally annealed pyrolytic graphite (APG) materials or combinations thereof e.g. Cu-APG or Aluminium-APG interface plates.

Thermal interface points may be provided for example, at the transmission interface 120 region and via the bolt 140a and 140b, depicted in FIGS. 3(a) and 3(b). The entire PCB 130 depicted in FIGS. 3(a) and 3(b) is provided on a metal carrier/substrate that sinks heat from the microwave power devices provided in the PCB 130. The additional bulk metal work of the radiating structure 114 may also sink some of this heat. Embedded “copper coin” methods may be implemented, between the microwave power device and base of PCB 130 to get heat into the substrate from the PCB 130.

It will be understood that a power source is provided for the microwave power generator module to generate microwave power. The power source may be from a port or electrical power loom intended to power or communicate to peripherals or tools. Suitable power schemes are known in the art and are not discussed in further detail.

In further embodiments, the device may have a controller for controlling one or more operational parameters of the device. For example, system/applicator temperatures, forward and reflected power, duty cycle, antenna performance attributes or other relevant parameters may be controlled. A feedback mechanism may also be provided to control operational parameters based on feedback from the device. It may also access communications or networks to communicate with an external controller to provide feedback.

A skilled person will appreciate that variations of the enclosed arrangement are possible without departing from the invention. Accordingly, the above description of the specific embodiment is made by way of example only and not for the purposes of limitations. It will be clear to the skilled person that minor modifications may be made without significant changes to the operation described.

1. A radio frequency (RF) or microwave energy applicator device for applying radio frequency or microwave radiation to a target, the applicator comprising:
an energy generator module for generating RF or microwave energy, wherein the energy generator module comprises an energy output for outputting said generated energy;
a radiating structure for radiating RF or microwave radiation to the target wherein the radiating structure comprises an energy input,
wherein the energy generator module and the radiating structure are coupled to provide the energy output of the energy generator module and the energy input of the radiating structure at a transmission interface;
wherein the transmission interface comprises at least one transmission feature comprising at least one of a size, dimension or shape selected so that at least part of the energy provided to the transmission interface is at least one of transmitted to the radiating structure or reflected.

2. The device as claimed in claim 1, wherein the energy output of the energy generator module and the energy input of the radiating structure are coupled such that no variable structure is required for tuning between the energy output of the energy generator and the energy input of the radiating structure.

3. The device as claimed in claim 1, wherein the transmission feature comprises a mismatch between the energy output of the energy generator module and the energy input of the radiating structure thereby to introduce a transmission inefficiency between the energy output of the energy generator and the energy input of the radiating structure.

4. The device as claimed in claim 1, wherein the radiating structure is a rigid structure and the energy generator module is a rigid structure and wherein the radiating structure and the energy generator module are rigidly coupled together.

5. The device as claimed in claim 1, wherein the radiating structure comprises a radiating surface from which radiation is emitted and wherein the transmission interface provides the only interface between the energy generator module and the radiating surface.

6. The device as claimed in claim 1, wherein at least one of the energy output of the energy generator module and the energy input of the radiating structure is at least one of shaped or sized to form the transmission feature at the transmission interface.

7. The device as claimed in claim 1, wherein the at least one of a size, dimension or shape is selected at least one of to substantially maximise a measure of transmitted power from the energy generator module to the radiating structure or to substantially minimize a transmission loss through the transmission interface.

8. The device as claimed in claim 1, wherein at least one of:
a) at least one design parameter for at least one of the radiating structure or the energy generator module is selected together with the at least one of size, dimension or shape of the transmission feature to provide a desired degree of impedance match between the energy output and the energy input, optionally, wherein at least one of the impedance of the energy output or the impedance of the energy input does not correspond to a standard impedance value, for example, an impedance value of 50Ω; or
b) at least one of at least one design parameter of the radiating structure or energy generator module at least one of is selected to provide a substantially simultaneous impedance match between the radiating structure and a desired surface and between the radiating generator module and the energy generator module or is selected such that, together with the transmission feature, a substantially system-wide conjugate match is achieved.

9. (canceled)

10. The device as claimed in claim 8, wherein the at least one design parameter comprises at least one of:
a) a dimension, for example, a height, width, length or thickness of at least part of the energy generator module, for example, the energy output;
b) a dimension, for example, a height, width, length or thickness of the radiating structure, for example the energy input;
c) a length of the exposed distal portion of a conductor of the energy input or output;
d) a length or phase property of the radiating structure;
e) an offset distance between parts of the radiating structure; or
f) a gap between a radiating element of the radiating structure and an outer conductor.

11. The device as claimed in claim 1, wherein the transmission feature comprises an overlapping feature, for example, a step feature, such that at least part of the energy output and at least part of the energy input are at least closely coupled along an overlap length, optionally wherein the portion of at least one of energy transmitted or energy reflected is in dependence on the overlap length.

12. The device as claimed in claim 11, wherein the overlap length is in the range 1 mm to 8 mm, in particular in the range 3 mm to 6 mm.

13. The device as claimed in claim 1, wherein at least one of:
a) the transmission interface comprises an interface between a microstrip structure and a co-axial structure;
b) at least one of the energy input of the radiating structure or the energy output of the energy generator module comprises a microstrip structure comprising a microstrip conductive element on a substrate; or
c) at least one of the energy input or the energy output of the radiating structure comprises a coaxial input structure comprising an inner conductor and an outer conductor.

14. The device as claimed in claim 1, wherein at least one of:
a) the energy output of the energy generator module comprises a first exposed length of a microstrip conductive element on a substrate and the energy input of the radiating structure comprises a second exposed length of an inner conductor of a coaxial structure such that when coupled, the first exposed length is provided at the second exposed length; or
b) wherein the energy generator module comprises a feedback mechanism configured to receive energy reflected by the radiating structure or a signal representative thereof and wherein one or more design parameters of the radiating structure is selected such that the radiating structure reflects a desired portion of energy provided to so that feedback mechanism causes the energy generator module to generate more energy.

15. (canceled)

16. The device as claimed in claim 1, wherein at least one of at least part of the energy generator module or at least part of the radiating structure is at least one of sized or shaped to fit the energy generator module together with the radiating structure such that, when fitted together, a conductive path is provided between the energy generator module and the radiating structure.

17. The device as claimed in claim 16, wherein the transmission feature further comprises an insulating portion at least partially surrounding the at least one conductive path, wherein the insulating portion is provided by at least one of at least part of the energy generator module or at least part of the radiating structure.

18. The device as claimed in claim 1, further comprising a coupling mechanism for coupling the energy generator module and the radiating structure and wherein the coupling mechanism provides at least one of:
a) at least one electrical path between the radiating structure and a ground of the energy generator module via a portion of the coupling mechanism; or
b) a first conductive path at an upper surface of the microwave generating module and a second conductive path at a lower surface of the microwave generating module.

19. (canceled)

20. (canceled)

21. (canceled)

22. The device as claimed in claim 1, wherein at least one of:
a) the radiating structure comprises any suitable antenna, for example, a dipole antenna, a monopole antenna, a horn, a waveguide;
b) the device further comprises a housing;
c) the energy generator module comprises an amplifier stage and wherein the transmission interface comprises a secondary coupling between the power amplifier of the generator module and the radiating structure;
d) the radiating structure comprises a second order extracted pole unit (EPU) composed of a pair of mutual coupled resonant elements;
e) the radiating structure comprises one or more dissipative elements configured to dissipate excess heat into metallic or thermally conductive elements within the radiating structure; or
f) the device further comprises a controller to control one or more operational parameters.

23. A method of designing a RF or microwave energy applicator device comprising:
generating a model representative of at least a transmission interface between an energy generator module and a radiating structure, wherein the transmission interface comprises at least one transmission feature;
varying one or more parameters representative of at least one of the size, dimension or shape parameters of at least the at least one transmission feature to determine changes in the portion of at least one of energy reflected or energy transmitted at the transmission interface;
selecting values for the one or more design parameters corresponding to a desired portion of at least one of energy reflected or energy transmitted via the transmission interface.

24. The method as claimed in claim 23, further comprising generating at least one further model representative of the interface between the radiating structure and a desired surface and combining the at least one further model with the model representative of at least the transmission interface, and selecting one or more design parameters of the radiating structure, the energy generator module and the transmission interface based on the combined model.

25. A method of manufacturing a RF or microwave energy applicator device comprising providing an energy generator module comprising an energy output and a radiating structure comprising an energy input in accordance with one or more design parameters such that the energy generator module and the radiating structure comprises one or more of at least one of transmission properties or reflection properties such that when the energy input and the energy output are coupled at a transmission interface, one or more transmission feature comprising at least one of a size, dimension or shape selected to at least one of transmit or reflect a desired portion of microwave energy provided to the one or more transmission feature from the energy generator module.
